(19) United States
(12) Patent Application Publication
(10) Pub. No.: US 2006/0251597 A1
(43) Pub. Date: Nov. 9, 2006

(54) ANTI-ODOR COMPOSITIONS AND THERAPEUTIC USE

(76) Inventors: Ruey J. Yu, Chalfont, PA (US); Eugene J. Van Scott, Abington, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/381,374

(22) Filed: May 3, 2006

Related U.S. Application Data
(60) Provisional application No. 60/677,364, filed on May 4, 2005.

(51) Int. Cl. A61L 9/00; A61K 8/46; A61K 8/49

(52) U.S. Cl. 424/65; 424/76.2

(57) ABSTRACT

This application discloses a composition comprising a malodor compound and an anti-odor ingredient effective for reducing the presence or production of malodor. The composition may be topically applied to a subject and is useful for cosmetic conditions, pharmaceutical indications, or other objectives.

This application claims priority under 35 U.S.C. §119 to Provisional Patent Application No. 60/667,364 filed on May 4, 2005.

### FIELD OF THE INVENTION

This application relates to compositions comprising a malodor compound useful for cosmetic conditions, pharmaceutical indications, or other objectives.

### BACKGROUND OF THE INVENTION

Many cosmetic and pharmaceutical substances comprising molecules having a thiol, mercaptan, or a like group have an inherent, unpleasant malodor. The malodor is objectionable to consumers when these substances are used as ingredients in topical compositions. It would thus be desirous to find a substance that can effectively prevent the malodor formation or to formulate a novel composition that, while containing a thiol or similar malodor compounds, does not have the malodor.

U.S. Pat. No. 5,733,535 entitled “Topical Compositions Containing N-Acetylcysteine and Odor Masking Materials” disclosed compositions comprising certain perfume chemicals to mask malodors associated with 0.01% to 50% N-acetylcysteine or related compounds. These perfume chemicals are selected from the group consisting of aromatic esters, aliphatic esters, aromatic alcohols, aliphatic alcohols, aliphatic ketones, aromatic aldehydes, aliphatic aldehydes, aromatic ethers, and aliphatic ethers at a combined concentration from 0.01% to 0.5%. The masking of N-acetylcysteine malodors by such perfume chemicals, however, has been found to be incomplete or ineffective due to different vapor pressure, different skin penetration rate, and a poor binding affinity between N-acetylcysteine and the perfume chemicals, especially when the composition is topically applied to human skin.

U.S. Pat. No. 6,905,675 B2 describs the adjustment of pH between 6.5 to pH 8.1 to control malodor of dermatological compositions containing sulfur compounds. The malodor control by adjusting the pH of a composition, however, is incomplete, unreliable, and ineffective, especially when the composition is topically applied to the skin due to the acidic pH of the skin surface,

U.S. Pat. No. 4,388,301 describes the use of montmorillonite clays, such as bentonites, kaolin, hectorite, smectite, saponite, and attapulgite to control the malodor of inorganic polysulfides, such as calcium polysulfide. The composition formed by use of the these montmorillonite clays is a paste composition that is messy when topically applied to the skin and fails to completely control the malodor produced by compositions comprising polysulfides or a sulfurated lime solution with pungent rotten egg odor.

The description herein of disadvantages and problems associated with known compositions, and methods is in no way intended to limit the scope of the embodiments described in this document to their exclusion. Indeed, aspects of the invention may include certain features of the described products, methods, and/or apparatus without suffering from their described disadvantages.

### SUMMARY OF THE INVENTION

It is a feature of an embodiment of the invention to provide compositions comprising a sulfone and/or organic amine having an amino, imino, and/or guanido group that can effectively prevent malodor produced by organic thiols or similar malodor compounds. It is a feature of an embodiment of the invention to provide a novel anhydrous composition comprising a thiol or similar malodor compounds that does not produce unpleasant malodor. We have found that whereas common solvents, such as water and ethanol, can enhance, but cannot suppress the malodor produced by a thiol or similar malodor compounds, certain other organic solvents and ingredients may effectively prevent the malodor formation in an anhydrous composition.

### DETAILED DESCRIPTION OF PREFERRED EMBODIMENTS

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a references to “a composition” includes a plurality of such compositions, as well as a single composition, and a reference to “a topically active agent” or a “malodor compound” is a reference to one or more agents and/or compounds, as well as equivalents thereof known to those skilled in the art, and so forth.

We have now discovered that sulfones, organic amines (preferably heterocyclic amines), solvents, and other ingredients can effectively prevent, counteract, or eliminate unpleasant malodor produced by malodor compounds. Sulfones and organic amines are particularly effective in reducing the malodor present or produced by an organic thiol or similar malodor compounds in a composition containing water or ethanol. For example, we have now discovered that dimethyl sulfone or methenamine can effectively prevent or eliminate the malodor produced by compounds like N-acetylcysteine. Thus, the sulfones or organic amines according to embodiments may be used to prevent or inhibit, for example, the malodor generated by a hair waving thiol or similar malodor compounds, such as thioglycolate.

The unpleasant or malodor compounds referred to in this description preferrably include, but are not limited to, organic mercaptans, thiol, and dithiol compounds and derivatives thereof. As used herein, a thiol is a compound that contains the functional group composed of a sulfur atom and a hydrogen atom (—SH). This functional group is referred to either as a thiol group or a sulfhydryl group. Thiols also may be referred to as mercaptans. Some specific examples of mercaptans, thiols, dithiols, and similar malodor compounds include, but are not limited to, glyceryl monomercaptan, thioglycolic acid, cysteine, cysteine esters, N-acetyl-cysteine, N-acetyl-cysteine esters; N,S-diacetyl-cysteine esters, glutathione, glutathione esters, N-acetyl-glutathione, N-acetyl-glutathione esters, methionine, ammonium thioglycolate, calcium thioglycolate, zinc thioglycolate, potassiumm thioglycolate, monoethanolammonium thioglycolate, mercaptopurine, lipoic acid, and 6,8-dimercaptooctanoic acid (dihydrolipoic acid). The malodors produced by these compounds range in degrees from rotten eggs, decomposed fish, dead animals, to substances ejected by a skunk.

As an illustration, N-acetylcysteine is a potent antioxidant, a precursor for biosynthesis of glutathione, and is therapeutically effective for topical prevention or treatment of various cosmetic conditions and dermatological disorders, such as severe dry skin, ichthyosis, age spots, melasma, lentigines, and skin changes associated with aging Therapeutic concentrations of N-acetylcysteine range from about 5% to about 10% in solution, oil-in-water lotion, or cream. These compositions, however, have a very unpleasant malodor similar to a rotten egg. Users of topical compositions containing N-acetylcysteine may develop resistance or become accustomed to the malodor, but the family members or bystanders may be disturbed by the objectionable and unpleasant odor of N-acetylcysteine released as vapor pressure in the air. Because of such shortcomings, N-acetylcysteine has very limited commercial utilization for topical administration to human skin, nails, or hair. Dimethyl sulfone or methenamine for example, can effectively prevent or eliminate the malodor produced by N-acetylcysteine in a composition containing water.

Without being limited to a theory, it is believed that the sulfone or organic amine may trap or bind a thiol or similar malodor compounds to form a molecular complex through ionic/ionic, ionic/dipolar, and/or dipolar/dipolar bonds that reduces the vapor pressure of the thiol or like substance. The effect of these anti-odor ingredients is thus to reduce the presence or prevent the production of malodor, and is not a masking effect. The inventors have found that the anti-odor effect of the anti-odor ingredients according to embodiments of the present invention is unrelated to the pH of the composition, and that the anti-odor effect is not a relatively masking effect produced by a perfume or fragrant substance. In fact, the preferred pH of a composition is on the acidic side ranging from about pH 3.0 to about pH 6.0, so that the active substance, such as N-acetyl-cysteine, has an optimal bioavailability to penetrate into the skin with minimal or no skin irritations. The compositions of the present embodiments have a pH of about 3.0 to about pH 6.5, preferably a pH of about 3.0 to about pH 6.0, and more preferably a pH of about 3.5 to about pH 5.5.

The sulfones according to preferred embodiments include sulfonyl compounds, which may be represented by the following generic structure:
R₁SO₂R₂,

where R₁ and R₂ are independently NH₂, NHCONH₂, NHC(═NH)NH₂, an alkyl, aralkyl, or aryl group having 1 to 14 carbon atoms; and R₁ and R₂ may also carry OH, NH₂, CONH₂, COOR₃, NHCONH₂, NHC(═NH)NH₂, imidazole, pyrrolidine, or other heterocyclic group; R₃ is H, an alkyl, aralkyl or aryl group having 1 to 9 carbon atoms; and the H attached to any carbon atom may be substituted by I, F, Cl, Br, OH or alkoxy group having 1 to 9 carbon atoms.

Examples of sulfones according to preferred embodiments include, but are not limited to, dimethyl sulfone, diethyl sulfone, diphenyl sulfone, methyl phenyl sulfone, dapsone; 4,4′-sulfonylbis(methylbenzoate); 4,4′-sulfonylbis(2-methylphenol); 4,4′-sulfonyldiphenol, 2,2′-sulfonyldiethanol, and mixtures thereof.

The organic amines of preferred embodiments are odorless and have at least one amino, imino and/or guanido group. More preferably, the organic amines are heterocyclic amines, which also may comprise other groups such as NHOH, NHNH₂, NHCONH₂, NHC(═NH)NH₂, CONH₂, alkoxyl, imidazole, pyrrolidine, or other heterocyclic ring. The inventors have found that heterocyclic amines, such as methenamine, are efficient in preventing or inhibiting the malodor produced by an organic thiol or similar malodor compounds, such as N-acetylcysteine.

The organic amines of preferred embodiments include, for example, methenamine. Methenamine, also known as hexamethylenetetramine, is a white, odorless heterocyclic compound with a sweet taste, having the formula C₆H₁₂N₄ and a molecular weight of 140. Methenamine in combination with mandelic acid, known as mandelamine, may be used orally to treat urinary infections. Glucamine and Meglumine (N-methyl-glucamine) are aminocarbohydrates.

Other organic amines according to the present embodiments include, but are not limited to, adenine, amantadine (1-adamantanamine), 2-adamantylamine, adenosine, agmatine, agroclavine, 1-(2-aminoethyl)piperazine, 5-amino-4-imidazolecarboxamide, 2-aminoimidazole, 2-amino-2-methyl-1-propanol, 2-aminoperimidine, 3-aminoquinuclidine, 4-amino-2,2,6,6-tetramethylpiperidine, 5-aminotetrazole, benzoctamine, benztropine, 1-benzylpiperazine; 4,4-bipiperidine chlorhexidine, cocamine, creatinine, cytidine, cytosine, dicocamine, diethanolamine, diisopropanolamine, dimethyl behenamine, dimethyl cocamine, edamine, glucamine, guanine, guanosine, meglumine (N-methylglucamine), memantine, methenamine, morpholine, oleamine, piperazine, porphine, quinuclidine, 3-quinuclidinol, quinupramine, rimantadine, soyamine, soyaminopropylamine, triethylenediamine, trizma base [tris(hydroxymethyl)aminomethane], tropane, tromantadine, tromethamine (2-amino-2-hydroxymethyl-1,3-propanediol), tropine, and tropinone.

Preferred organic amines include amantadine, 1-(2-aminoethyl)piperazine, chlorhexidine, glucamine, meglumine (N-methylglucamine), memantine, methenamine, piperazine, porphine, quinuclidine, 3-quinuclidinol, quinupramine, rimantadine, triethylenediamine, tropane, tromantadine, tropine, and tropinone.

Many organic amines from commercial sources are in salt form, such as chlorhexidine gluconate and amantadine hydrochloride. Such salt forms have been found to be less effective in preventing the malodor produced by thiol compounds. Therefore, the salt form of an organic amine may be converted to a free base form by reaction with an inorganic alkali before use as an anti-odor substance.

A thiol or similar malodor compounds used in the formulation of a cosmetic or pharmaceutical composition may range in concentration, for example, from about 1% to about 50% by weight of the total composition. The concentration of a sulfone or organic amine of the present embodiments used to prevent the malodor formation by a thiol or similar malodor compounds may range from about 0.1% to about 20% by weight of the total composition, and preferably from about 1% to about 20% by weight of the total composition.

The inventors also have discovered that a novel anhydrous composition comprising a thiol or similar malodor compounds without the sulfone or organic amine can effectively prevent the malodor formation. The inventors believe that whereas common solvents such as water, ethanol, propylene glycol and butylene glycol may enhance or may not suppress malodor production by a thiol or similar malodor compounds, certain other solvents or ingredients such as isopropyl alcohol, n-propanol, ethyl acetoacetate, alpha-hydroxyacid esters (such as diethyl tartrate and triethyl citrate), and N-acetylamino acid esters (such as N-acetyl-L-proline ethyl ester and N-acetyl-L-proline propyl ester) may effectively prevent malodor formation. The concentration of these solvents or ingredients in an anhydrous composition may be about 1% or higher concentration by weight.

Representative solvents and other ingredients that may be used to prevent or suppress malodor formation by a thiol or similar malodor compounds in an anhydrous composition include, but are not limited to, the following alone or in various combination with one another:

(1) Alpha-Hydroxyacid Esters

(a) methyl glycolate, methyl lactate, methyl 2-methyllactate, methyl 2-hydroxybutanoate, methyl 2-hydroxypentanoate, methyl 2-hydroxyhexanoate, methyl 2-hydroxyheptanoate, methyl 2-hydroxyoctanoate, methyl 2-hydroxyeicosanoate, methyl cerebronate, methyl 2-hydroxynervonate, methyl mandelate, methyl benzilate, methyl 3-phenyllactate, methyl atrolactate, dimethyl malate, dimethyl tartarate, trimethyl citrate, trimethyl isocitrate, methyl 3-hydroxypropanoate, methyl 3-hydroxybutanoate, methyl 3-hydroxypentanoate and methyl tropate;

(b) ethyl glycolate, ethyl lactate, ethyl 2-methyllactate, ethyl 2-hydroxybutanoate, ethyl 2-hydroxypentanoate, ethyl 2-hydroxyhexanoate, ethyl 2-hydroxyheptanoate, ethyl 2-hydroxyoctanoate, ethyl 2-hydroxyeicosanoate, ethyl cerebronate, ethyl 2-hydroxynervonate, ethyl mandelate, ethyl benzilate, ethyl 3-phenyllactate, ethyl atrolactate, diethyl malate, diethyl tartarate, triethyl citrate, triethyl isocitrate, ethyl 3-hydroxypropanoate, ethyl 3-hydroxybutanoate, ethyl 3-hydroxypentanoate and ethyl tropate;

(c) propyl glycolate, propyl lactate, propyl 2-methyllactate, propyl 2-hydroxybutanoate, propyl 2-hydroxypentanoate, propyl 2-hydroxyhexanoate, propyl 2-hydroxyheptanoate, propyl 2-hydroxyoctanoate, propyl 2-hydroxyeicosanoate, propyl cerebronate, propyl 2-hydroxynervonate, propyl mandelate, propyl benzilate, propyl 3-phenyllactate, propyl atrolactate, dipropyl malate, dipropyl tartarate, tripropyl citrate, tripropyl isocitrate, propyl 3-hydroxypropanoate, propyl 3-hydroxybutanoate, propyl 3-hydroxypentanoate, and propyl tropate; and

(d) isopropyl glycolate, isopropyl lactate, isopropyl 2-methyllactate, isopropyl 2-hydroxybutanoate, isopropyl 2-hydroxypentanoate, isopropyl 2-hydroxyhexanoate, isopropyl 2-hydroxyheptanoate, isopropyl 2-hydroxyoctanoate, isopropyl 2-hydroxyeicosanoate, isopropyl cerebronate, isopropyl 2-hydroxynervonate, isopropyl mandelate, isopropyl benzilate, isopropyl 3-phenyllactate, isopropyl atrolactate, diisopropyl malate, diisopropyl tartarate, triisopropyl citrate, triisopropyl isocitrate, isopropyl 3-hydroxypropanoate, isopropyl 3-hydroxybutanoate, isopropyl 3-hydroxypentanoate, and isopropyl tropate.

(2) N-Acetylamino Acid Esters

(a) methyl N-acetyl-alaninate, methyl N-acetyl-argininate, methyl N-acetyl-asparaginate, dimethyl N-acetyl-aspartate, methyl N-acetyl-glycinate, dimethyl N-acetyl-glutamate, methyl N-acetyl-glutaminate, methyl N-acetyl-histidinate, methyl N-acetyl-isoleucinate, methyl N-acetyl-leucinate, methyl N-acetyl-lysinate, methyl N-acetyl-phenylalaninate, methyl N-acetyl-prolinate, methyl N-acetyl-serinate, methyl N-acetyl-threoninate, methyl N-acetyl-tryptophanate, methyl N-acetyl-tyrosinate, methyl N-acetyl-valinate, methyl N-acetyl-β-alaninate, methyl N-acetyl-γ-aminobutanoate, methyl N-acetyl-β-aminoisobutanoate, methyl N-acetyl-citruilinate, methyl N-acetyl-homoserinate, methyl N-acetyl-ornithinate, methyl N-acetyl-phenylglycinate, methyl N-acetyl-4-hydroxyphenylglycinate, and methyl N,O-diacetyl-4-hydroxyphenylglycinate;

(b) ethyl N-acetyl-alaninate, ethyl N-acetyl-argininate, ethyl N-acetyl-asparaginate, diethyl N-acetyl-aspartate, ethyl N-acetyl-glycinate, diethyl N-acetyl-glutamate, ethyl N-acetyl-glutaminate, ethyl N-acetyl-histidinate, ethyl N-acetyl-isoleucinate, ethyl N-acetyl-leucinate, ethyl N-acetyl-lysinate, ethyl N-acetyl-phenylalaninate, ethyl N-acetyl-prolinate, ethyl N-acetyl-serinate, ethyl N-acetyl-threoninate, ethyl N-acetyl-tryptophanate, ethyl N-acetyl-tyrosinate, ethyl N-acetyl-valinate, ethyl N-acetyl-β-alaninate, ethyl N-acetyl-γ-aminobutanoate, ethyl N-acetyl-β-aminoisobutanoate, ethyl N-acetyl-citrullinate, ethyl N-acetyl-homoserinate, ethyl N-acetyl-ornithinate, ethyl N-acetyl-phenylglycinate, ethyl N-acetyl-4-hydroxyphenylglycinate, and ethyl N,O-diacetyl-4-hydroxyphenylglycinate;

(c) propyl N-acetyl-alaninate, propyl N-acetyl-argininate, propyl N-acetyl-asparaginate, dipropyl N-acetyl-aspartate, propyl N-acetyl-glycinate, dipropyl N-acetyl-glutamate, propyl N-acetyl-glutaminate, propyl N-acetyl-histidinate, propyl N-acetyl-isoleucinate, propyl N-acetyl-leucinate, propyl N-acetyl-lysinate, propyl N-acetyl-phenylalaninate, propyl N-acetyl-prolinate, propyl N-acetyl-serinate, propyl N-acetyl-threoninate, propyl N-acetyl-tryptophanate, propyl N-acetyl-tyrosinate, propyl N-acetyl-valinate, propyl N-acetyl-β-alaninate, propyl N-acetyl-γ-aminobutanoate, propyl N-acetyl-β-aminoisobutanoate, propyl N-acetyl-citruilinate, propyl N-acetyl-homoserinate, propyl N-acetyl-ornithinate, propyl N-acetyl-phenylglycinate, propyl N-acetyl-4-hydroxyphenylglycinate, and propyl N,O-diacetyl-4-hydroxyphenylglycinate; and

(d) isopropyl N-acetyl-alaninate, isopropyl N-acetyl-argininate, isopropyl N-acetyl-asparaginate, diisopropyl N-acetyl-aspartate, isopropyl N-acetyl-glycinate, diisopropyl N-acetyl-glutamate, isopropyl N-acetyl-glutaminate, isopropyl N-acetyl-histidinate, isopropyl N-acetyl-isoleucinate, isopropyl N-acetyl-leucinate, isopropyl N-acetyl-lysinate, isopropyl N-acetyl-phenylalaninate, isopropyl N-acetyl-prolinate, isopropyl N-acetyl-serinate, isopropyl N-acetyl-threoninate, isopropyl N-acetyl-tryptophanate, isopropyl N-acetyl-tyrosinate, isopropyl N-acetyl-valinate, isopropyl N-acetyl-β-alaninate, isopropyl N-acetyl-γ-aminobutanoate, isopropyl N-acetyl-β-aminoisobutanoate, isopropyl N-acetyl-citrullinate, isopropyl N-acetyl-homoserinate, isopropyl N-acetyl-ornithinate, isopropyl N-acetyl-phenylglycinate, isopropyl N-acetyl-4-hydroxyphenylglycinate, and isopropyl N,O-diacetyl-4-hydroxyphenylglycinate.

(3) Other Ingredients

(a) butoxydiglycol, butoxyethanol, butoxypropanol, butyl acetate, butanol, butyloctanol, cycloethoxymethicone, cyclohexane, cyclomethicone, dibutyl adipate, diisobutyl adipate, diisopropyl adipate, diisopropyl oxalate, diisopropyl sebacate, dimethicone, dimethoxydiglycol, dimethyl adipate, dimethyl glutarate, dioctyl adipate, dioctyl sebacate, dioctyl succinate, dipropyl adipate, dipropylene glycol, ethoxydiglycol, ethoxyethanol, 2-ethyl-1,3-hexanediol, isododecane, isooctane, isoparaffin, isopropyl acetate, isopropyl alcohol, isopropyl myristate, isopropyl palmitate, methicone, octadecane, octanol, octyl benzoate, oleyl alcohol, oleyl lactate, PEG, phenyl methicone, n-propanol, ethyl acetoacetate, methyl acetoacetate, propyl acetoacetate, isopropyl acetoacetate, and silicone oil.

If an anhydrous composition is used for cosmetic conditions or pharmaceutical indications, a thiol or similar malodor compounds used in the formulation may range in concentration from about 1% to about 50% by weight of the total composition, for example. A solvent or ingredient of the present embodiments used to prevent the malodor formation by a thiol or similar malodor compounds may be present in a composition at a concentration from about 1% or higher concentration by weight of the total composition, preferably from about 1% to about 90% by weight of the total composition, and more preferably from about 5% to about 80% by weight of the total composition.

The preferred solvents or ingredients that may be used to prevent or suppress malodor formation by a thiol or similar malodor compounds in an anhydrous composition are benzyl N-acetyl-prolinate, ethyl N-acetyl-prolinate, propyl N-acetyl-prolinate, isopropyl N-acetyl-prolinate, diethyl tartrate, diisopropyl adipate, ethoxydiglycol, ethyl glycolate, ethyl lactate, ethyl acetoacetate, isopropyl alcohol, isopropyl myristate, isopropyl palmitate, oleyl lactate, n-propanol, silicone oil, triethyl citrate, tripropyl citrate, and triisopropyl citrate.

According to the present embodiments, there are two preferred options to formulate a composition to prevent or suppress malodor formation by a thiol or similar malodor compounds. One is to use a sulfone or organic amine to suppress or prevent the malodor formation in a composition that contains water or alcohol. The other option is to formulate an anhydrous composition containing a certain solvent or ingredient.

The degree of reduction in the presence or production of malodor produced by a thiol or similar malodor compounds in a composition comprising a sulfone or organic amine was judged by a three-member panel through olfactory sensor by sniffing as (a) no difference from control, (b) slight malodor, (c) trace malodor, and (d) no malodor. Initially, a malodor compound such as about 5% by weight was prepared in water solution or in oil-in-water emulsion. A sulfone or organic amine of the present embodiments was added at various concentrations for example, ranging from about 2%, 1%, 0.5% and 0.1% to the formulations containing the malodor substance. The composition containing the malodor substance without a sulfone or organic amine was used as the control. The test compositions and control were judged by the panel for the anti-odor effect and the degree of reducing the presence or preventing the production of malodor was recorded as (a) no effect (no difference from control), (b) slight malodor (50% effective), (c) trace malodor (90% effective), and (d) no malodor (completely effective). As an illustration, methenamine at 0.1% or higher concentration can completely eliminate the malodor produced by N-acetyl-L-cysteine 2% in oil-in-water emulsion.

In the same manner, the degree of reducing the presence or preventing the production of malodor generated by a thiol or similar malodor compounds in an anhydrous composition containing a solvent or ingredient of the present embodiments was judged by three-member panel through olfactory sensor by sniffing as (a) no difference from control, (b) slight malodor, (c) trace malodor, and (d) no malodor. In an initial test, a solvent or liquid ingredient of the present embodiments at 4.5 ml was added to a test tube, and a thiol or similar malodor compounds 0.5 g was added to make 10% thiol or similar malodor compounds in the solution. The control solution was prepared by dissolving a thiol or similar malodor compounds 0.5 g in water 4.5 ml to make 10% aqueous solution. The above test solutions including the control solutions were judged by the panel for the anti-odor effect and the degree of reducing the presence or preventing the production of malodor was recorded in the same way as (a) no effect (no difference from control), (b) slight malodor (50% effective), (c) trace malodor (90% effective), and (d) no malodor (completely effective).

Based on the above tests, the following solvents or ingredients may be used to effectively prevent malodor produced by a thiol or similar malodor compounds: benzyl N-acetyl-prolinate, ethyl N-acetyl-prolinate, propyl N-acetyl-prolinate, isopropyl N-acetyl-prolinate, butoxydiglycol, butoxyethanol, butoxypropanol, butyl acetate, N-butyl alcohol, butylene glycol, butyl lactate, butyloctanol, cycloethoxymethicone, cyclohexane, cyclomethicone, dibutyl adipate, diethyl tartrate, diisobutyl adipate, diisopropyl adipate, diisopropyl oxalate, diisopropyl sebacate, dimethicone, dimethoxydiglycol, dimethyl adipate, dimethyl glutarate, dimethyl sulfone, dioctyl adipate, dioctyl sebacate, dioctyl succinate, dipropyl adipate, dipropylene glycol, ethoxydiglycol, ethoxyethanol, ethyl glycolate, 2-ethyl-1,3-hexanediol, ethyl lactate, ethyl acetoacetate, isododecane, isooctane, isoparaffin, isopropyl acetate, isopropyl alcohol, isopropyl myristate, isopropyl palmitate, methicone, octadecane, octanol, octyl benzoate, oleyl alcohol, oleyl lactate, PEG, phenyl methicone, n-propanol, silicone oil, triethyl citrate, triisopropyl citrate, and tripropyl citrate.

In a combination composition, a cosmetic, pharmaceutical, or other topically active agent may be incorporated alone or in combination with one another into the composition comprising an anti-odor ingredient and/or a malodor compound according to embodiments of the present invention. These topical agents may include, but are not limited to: hydroxyacids, ketoacids and related compounds; phenyl alpha acyloxyalkanoic acids and derivatives; N-acetyl-aidosamines, N-acetylamino acids and related N-acetyl compounds; local analgesics and anesthetics; anti acne agents; antibacterials; antiyeast agents; antifungal agents; antiviral agents; antidandruff agents; antidermatitis agents; antihistamine agents; antipruritic agents; antiemetics; antimotion sickness agents; antiinflammatory agents; antihyperkeratotic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; astringents; cleansing agents; corn, callus and wart removing agents; topical cardiovascular agents; vitamins; corticosteroids; tanning agents; hormones; retinoids; and gum disease or oral care agents.

Examples of other topically active agents that may be added to the compositions of the present embodiments include, but are not limited to: abacavir, acebutolol, acetaminophen, acetaminosalol, acetazolamide, acetohydroxamic acid, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydrornorphine, diltiazem, diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofioxacin, lidocaine, linezolid, lobetine, loperamide, losartan, loxapine, lysergic diethylamide, mafenide, malic acid, mattobionic acid, mandelic acid, maprotiline, mebendazole, mecamylamime, meclizine, meclocycline, memantine, menthol, meperidine, mepivacaine, mescaline, metanephrine, metaproterenol, metaraminol, metformrin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, pheneizine, pheniramine, phenmetrazine, phenobarbital, phenol, phenoxybenzamine, phentolamine, phenylephrine, phenylpropanolamine, phenytoin, physostigmine, pilocarpine, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, shale tar, sibutramine, sildenafil, sotalol, streptomycin, strychnine, sulconazole, sulfabenz, sulfabenzamide, sulfabromomethazine, sulfacetamide, sulfachiorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan, and zolpidem.

General Preparations

Compositions comprising a thiol or similar malodor compounds and a sulfone or organic amine of the embodiments may be formulated, for example, as solution, gel, lotion, cream, ointment, shampoo, spray, stick, powder, masque, mouth rinse or wash, vaginal gel or preparation, or other form acceptable for use on skin, nail, hair, oral mucosa, vaginal or anal mucosa, mouth or gums.

To prepare a solution composition, at least one sulfone or organic amine of the present embodiments and at least one thiol or similar malodor compounds are dissolved in a solution prepared from water, ethanol, propylene glycol, butylene glycol, and/or other topically acceptable vehicle. The concentration of the thiol or similar malodor compounds may range from about 0.1% to about 99% by weight of the total composition, with preferred concentration of from about 0.5% to about 70% by weight of the total composition and with more preferred concentration of from about 1% to about 50% by weight of the total composition. The concentration of the sulfone or organic amine may range from about 0.01% to about 90% by weight of the total composition, with preferred concentration of from about 0.05% to about 50% by weight of the total composition, and with more preferred concentration of from about 0.1% to about 20% by weight of the total composition.

To prepare a topical composition in lotion, cream or ointment form, the sulfone or organic amine of the present embodiments and a thiol or similar malodor compounds are dissolved first in water, ethanol, propylene glycol, and/or other topically acceptable vehicle, and the solution thus obtained is mixed with a desired base or pharmaceutically acceptable vehicle to make lotion, cream or ointment. The concentration of the thiol or similar malodor compounds may range from about 0.1% to about 99% by weight of the total composition, with preferred concentration of from about 0.5% to about 70% by weight of the total composition, and with more preferred concentration of from about 1% to about 50% by weight of the total composition. The concentration of the sulfone or organic amine may range from about 0.01% to about 90% by weight of the total composition, with preferred concentration of from about 0.05% to about 50% by weight of the total composition and with more preferred concentration of from about 0.1% to about 20% by weight of the total composition.

A topical composition also may be formulated into a gel or shampoo form. A typical gel composition is formulated by the addition of a gelling agent, such as chitosan, methyl cellulose, ethyl cellulose, polyvinyl alcohol, polyquaterniums, hydroxyethylcellulose, hydroxypropylcellulose, hydroxypropylmethylcellulose, carbomer, or ammoniated glycyrrhizinate, to a solution comprising a thiol or similar malodor compounds and a sulfone or organic amine of the present embodiments. The preferred concentration of the gelling agent may range, for example, from about 0.1% to about 2% by weight of the total composition. In the preparation of a shampoo, the organic amine of the present embodiments and the thiol or similar malodor compounds may be dissolved in water or propylene glycol, and the solution thus obtained is mixed with a shampoo base. The concentration of the thiol or similar malodor compounds may range from about 0.1% to about 99% by weight of the total composition, with preferred concentration of from about 0.5% to about 70% by weight of the total composition, and with more preferred concentration of from about 1% to about 50% by weight of the total composition. The concentration of the sulfone or organic amine may range from about 0.01% to about 90% by weight of the total composition, with preferred concentration of from about 0.05% to about 50% by weight of the total composition, and with more preferred concentration of from about 0.1% to about 20% by weight of the total composition.

To prepare an anhydrous composition according to the present embodiments, a thiol or similar malodor compounds may be dissolved in an organic solvent or ingredient of the present embodiments, and other solvents and ingredients (excluding water, ethanol, propylene glycol and butylene glycol) may be added to the solution. The concentration of the thiol or similar malodor compounds may range from about 0.1% to about 99% by weight of the total composition, with preferred concentration of from about 1% to about 70% by weight of the total composition and with more preferred concentration of from about 1% to about 50% by weight of the total composition. The concentration of each solvent or ingredient, or the combined total concentration of the solvents and ingredients of the present invention may range from about 1% to about 99% by weight of the total composition, with preferred concentration of from about 5% to about 85% by weight of the total composition, and with more preferred concentration of from about 10% to about 80% by weight of the total composition.

An anhydrous composition comprising a thiol or similar malodor compounds and a solvent or ingredient of the present embodiments also may be formulated by those skilled in the art as a gel, lotion, cream, ointment, shampoo, spray, stick, powder, masque or other form acceptable for use on skin, nail, hair, oral mucosa, vaginal or anal mucosa, mouth or gums. The concentration of the thiol or similar malodor compounds may range from about 0.1% to about 99% by weight of the total composition, with preferred concentration of from about 1% to about 70% by weight of the total composition and with more preferred concentration of from about 1% to about 50% by weight of the total composition. The concentration of each solvent or ingredient, or the combined total concentration of the solvents and ingredients of the present invention may range from about 1% to about 99% by weight of the total composition, with preferred concentration of from about 5% to about 85% by weight of the total composition, and with more preferred concentration of from about 10% to about 80% by weight of the total composition.

To prepare a combination, a cosmetic, pharmaceutical, or other topically active agent is incorporated into any one of the above compositions by dissolving or mixing the agent into the formulation. Other forms of compositions for topical delivery of active ingredients are readily blended, prepared or formulated by those skilled in the art.

Many thiols and similar malodor compounds, such as N-acetyl-cysteine, glutathione, and dihydrolipoic acid, are potent antioxidants and are topically effective for plumping the skin; for increasing skin thickness by stimulating biosynthesis of collagen and glycosaminoglycans; and for prevention or treatment of various cosmetic conditions, dermatological indications, and other disorders. These conditions, indications, and disorders include, for example, deranged or disordered cutaneous tissues relevant to skin, nail and hair; oral, vaginal and anal mucosa; promotion of wound healing; disturbed keratinization; inflammation; and changes associated with intrinsic and extrinsic aging. The manifestations of these conditions, indications, and disorders include, but are not limited to, acne; age spots; blemished skin; blotches; cellulite; dermatoses; dandruff; dry skin; eczema; elastosis; herpes; hyperkeratosis; hyperpigmented skin; severe dry skin, ichthyosis; keratoses; lentigines; melasmas; mottled skin; pseudofolliculitis barbae; photoaging and photodamage; pruritus; psoriasis; skin lines; stretch marks; thinning of skin, nail plate and hair; warts; wrinkles; xerosis; oral or gum disease; irritated, inflamed, unhealthy, damaged or abnormal mucosa, skin, hair, nail, nostril, ear canal, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing.

The anti-odor or malodor preventing composition according to the present embodiments may widen the horizon of applications and broaden the commercial utilities because the composition does not have unpleasant malodor. Thus, the present embodiments may be practiced in various ways by those skilled in the art. For example, instead of a single composition comprising both a malodor thiol compound and an anti-odor substance of the present embodiments, there may be two compositions; one comprises a thiol compound and the other an anti-odor substance. The two compositions may be topically applied to the skin or other site of the body simultaneously or alternatively in any order of application. In another example, an applicator may be utilized that contains two chambers or compartments; one comprising a malodor thiol compound either in powder or liquid form, and the other comprising an anti-odor substance of the present embodiments. When the applicator is squeezed or pumped, the compositions in the two chambers may be delivered simultaneously and/or mixed instantly before contacting or reaching the skin or other sites of the body. The above practices may serve to eliminate the malodor produced by the thiol or similar malodor compounds.

The examples that follow describe representative experiments conducted to identify compositions according to the present embodiments, which are useful, for example, in the topical prevention or treatment of various cosmetic conditions, dermatological indications, and other disorders, such as melasma, age spots and wrinkles. The following examples are illustrative, but not limiting, of the methods and compositions of the present embodiments. Other suitable modifications and adaptations of the variety of conditions and parameters normally encountered in the preparation and use of the compositions, and that are obvious to those skilled in the art, are within the spirit and scope of the embodiments.

### EXAMPLE 1

N-Acetyl-L-cysteine 10 g was dissolved in water 20 ml and the solution thus obtained was mixed with hydrophilic ointment or oil-in-water emulsion 70 g. The cream composition thus prepared contained 10% N-acetyl-L-cysteine and had very unpleasant malodor, as judged by three-member panel through olfactory sensor by sniffing.

N-Acetyl-L-cysteine 10 g and methenamine 3 g were dissolved in water 20 ml and the solution thus obtained was mixed with hydrophilic ointment or oil-in-water emulsion 67 g. The cream thus prepared contained 10% N-acetyl-L-cysteine and 3% methenamine, and the composition did not have any malodor as judged by the panel. This result shows that methenamine of the present embodiments can effectively eliminate or prevent the malodor of N-acetyl-L-cysteine in a cream or oil-in-water emulsion. The composition may be used, for example, in the topical prevention or treatment of various cosmetic conditions, dermatological indications and other disorders including, but not limited to, melasma, age spots and wrinkles.

### EXAMPLE 2

N-Acetyl-L-cysteine 10 g and meglumine (N-methyl-glucamine) 10 g were dissolved in water 20 ml and the solution thus obtained was mixed with hydrophilic ointment or oil-in-water emulsion 60 g. The cream thus prepared contained 10% N-acetyl-L-cysteine and 10% meglumine, and the composition had a trace of faint odor as judged by the panel. This result shows that meglumine of the present embodiments can substantially eliminate or prevent the malodor produced by N-acetyl-L-cysteine in a cream or oil-in-water emulsion. The composition may be used, for example, in the topical prevention or treatment of various cosmetic conditions, dermatological indications and other disorders including, but not limited to, melasma, age spots and wrinkles.

### EXAMPLE 3

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. Methenamine (MW 140) 7.0 g was dissolved in water, and the total volume was 100 ml. This solution contained methenamine 0.5M or 70 mg per ml methenamine.

In a first experiment, the above 0.5 M N-acetyl-L-cysteine 10 ml was mixed with 0.5 M methenamine 5 ml and water 5 ml. The final composition had pH 5.2, and contained N-acetyl-L-cysteine 4% and methenamine 1.8%. This composition did not have any malodor as judged by the panel. The composition may be used, for example, in the topical prevention or treatment of various cosmetic conditions, dermatological indications and other disorders including, but not limited to, melasma, age spots and wrinkles.

In a second experiment, the above 0.5 M N-acetyl-L-cysteine 25 ml was mixed with 0.5 M methenamine 5 ml and water 20 ml. The final composition had pH 3.9, and contained N-acetyl-L-cysteine 4% and methenamine 0.7%. This composition did not have any malodor as judged by the panel. The composition may be used, for example, in the topical prevention or treatment of various cosmetic conditions, dermatological indications and other disorders including, but not limited to, melasma, age spots and wrinkles.

These compositions may be used, for example, in the topical prevention or treatment of various cosmetic conditions, dermatological indications and other disorders including, but not limited to, melasma, age spots and wrinkles.

### EXAMPLE 4

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. Meglumine (N-methyl-D-glucamine, MW 195) 9.75 g was dissolved in water, and the total volume was 100 ml. This solution contained meglumine 0.5 M or 98 mg per ml meglumine.

In a first experiment, the above 0.5 M N-acetyl-L-cysteine 10 ml was mixed with 0.5 M meglumine 5 ml and water 5 ml. The final composition had pH 3.4 and contained N-acetyl-L-cysteine 4% and meglumine 2.5%. This composition had a trace malodor and was 90% effective in preventing odor as judged by the panel.

In a second experiment, the above 0.5 M N-acetyl-L-cysteine 25 ml was mixed with 0.5 M meglumine 5 ml and water 20 ml. The final composition had pH 2.9, and contained N-acetyl-L-cysteine 4% and meglumine 0.98%. This composition had a slight malodor and was 50% effective in preventing odor as judged by the panel.

These compositions may be used, for example, in the topical prevention or treatment of various cosmetic conditions, dermatological indications and other disorders including, but not limited to, melasma, age spots and wrinkles.

### EXAMPLE 5

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. Piperazine (MW 86) 4.3 g was dissolved in water, and the total volume was 100 ml. This solution contained piperazine 0.5M, or 43 mg per ml piperazine.

In a first experiment, the above 0.5 M N-acetyl-L-cysteine 10 ml was mixed with 0.5 M piperazine 5 ml and water 5 ml. The final composition had a pH 5.6, and contained N-acetyl-L-cysteine 4% and piperazine 1.1%. This composition had a slight malodor and was 50% effective in preventing odor as judged by the panel.

In a second experiment, the above 0.5 M N-acetyl-L-cysteine 25 ml was mixed with 0.5 M piperazine 5 ml and water 20 ml. The final composition had pH 3.4, and contained N-acetyl-L-cysteine 4% and piperazine 0.43%. This composition had a slight malodor and was 50% effective in preventing odor as judged by the panel.

These compositions may be used, for example, in the topical prevention or treatment of various cosmetic conditions, dermatological indications and other disorders including, but not limited to, melasma, age spots and wrinkles.

### EXAMPLE 6

An anti-odor test was carried out as follows: N-Acetyl-L-cysteine (163) 8.15 g was dissolved in water, and the total volume was 100 ml. This solution contained N-Acetyl-L-cysteine 0.5 M, or 81.5 mg per ml N-Acetyl-L-cysteine. Trizma base (MW 121) 6.05 g was dissolved in water, and the total volume was 100 ml. This solution contained trizma base 0.5M, or 60.5 mg per ml trizma base.

In a first experiment, the above 0.5 M N-acetyl-L-cysteine 10 ml was mixed with 0.5 M trizma base 5 ml and water 5 ml. The final composition had pH 3.5, and contained N-acetyl-L-cysteine 4% and trizma base 1.5%. This composition had a slight malodor and was 50% effective in preventing odor as judged by the panel.

In a second experiment, the above 0.5 M N-acetyl-L-cysteine 25 ml was mixed with 0.5 M trizma base 5 ml and water 20 ml. The final composition had pH 3.0, and contained N-acetyl-L-cysteine 4% and trizma base 0.61%. This composition had a slight malodor and was 50% effective in preventing odor as judged by the panel.

These compositions may be used, for example, in the topical prevention or treatment of various cosmetic conditions, dermatological indications and other disorders including, but not limited to, melasma, age spots and wrinkles.

### EXAMPLE 7

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. Chlorhexidine base (MW 505) 1.01 g was suspended in water, and the total volume was 100 ml. This suspension contained chlorhexidine base 0.2 M or 10.1 mg per ml chlorhexidine base.

The above 0.5 M N-acetyl-L-cysteine 5 ml was mixed with 0.2 M chlorhexidine base 5 ml. The final composition contained N-acetyl-L-cysteine 0.25 M and chlorhexidine 0.1 M. This composition had a slight malodor and was 50% effective in preventing odor as judged by the panel. The composition may be used, for example, in the topical prevention or treatment of various cosmetic conditions, dermatological indications and other disorders including, but not limited to, melasma, age spots and wrinkles.

### EXAMPLE 8

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. The above 0.5 M N-Acetyl-L-cysteine 50 ml was mixed with 30 ml water containing 1 g cytidine (MW 243), and the final volume was made up to 100 ml with water. This solution had pH 4.3 and contained 4% N-acetyl-L-cysteine and 1% cytidine. This composition had a trace malodor and was 90% effective in preventing odor as judged by the panel. This result shows that cytidine may substantially prevent the malodor produced by N-Acetyl-L-cysteine. The composition may be used, for example, in the topical prevention or treatment of various cosmetic conditions, dermatological indications and other disorders including, but not limited to, melasma, age spots and wrinkles.

### EXAMPLE 9

An anti-odor test was carried out as follows: N-Acetyl-L-cysteine (MW 163) 8.15 g was dissolved in water, and the total volume was 100 ml. This solution contained N-Acetyl-L-cysteine 0.5 M, or 81.5 mg per ml N-Acetyl-L-cysteine. The above 0.5 M N-Acetyl-L-cysteine 50 ml was mixed with 30 ml water containing 1 g cytosine (MW 111), and the final volume was made up to 100 ml with water. This solution contained 4% N-Acetyl-L-cysteine and 1% cyosine. This composition had a slight malodor and was 50% effective in preventing malodor as judged by the panel. The composition may be used, for example, in the topical prevention or treatment of various cosmetic conditions, dermatological indications and other disorders including, but not limited to, melasma, age spots and wrinkles.

### EXAMPLE 10

An anti-odor test was carried out as follows: N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. The above 0.5 M N-Acetyl-L-cysteine 50 ml was mixed with 30 ml water containing 1 g adenine (MW 135), and the final volume was made up to 100 ml with water. This solution contained 4% N-Acetyl-L-cysteine and 1% adenine. This composition had a slight malodor and was 50% effective in preventing malodor as judged by the panel. The composition may be used, for example, in the topical prevention or treatment of various cosmetic conditions, dermatological indications and other disorders including, but not limited to, melasma, age spots and wrinkles.

### EXAMPLE 11

An anti-odor test was carried out as follows: N-Acetyl-L-cysteine (MW 163) 8.15 g was dissolved in water, and the total volume was 100 ml. This solution contained N-Acetyl-L-cysteine 0.5 M, or 81.5 mg per ml N-Acetyl-L-cysteine. The above 0.5 M N-Acetyl-L-cysteine 50 ml was mixed with 30 ml water containing 1 g adenosine (MW 267), and the final volume was made up to 100 ml with water. This solution contained 4% N-Acetyl-L-cysteine and 1% adenosine. This composition had a slight malodor and was 50% effective in preventing malodor as judged by the panel. The composition may be used, for example, in the topical prevention or treatment of various cosmetic conditions, dermatological indications and other disorders including, but not limited to, melasma, age spots and wrinkles.

### EXAMPLE 12

An anti-odor test was also carried out as follows: N-Acetyl-L-cysteine (MW 163) 8.15 g was dissolved in water, and the total volume was 100 ml. This solution contained N-Acetyl-L-cysteine 0.5 M, or 81.5 mg per ml N-Acetyl-L-cysteine. The above 0.5 M N-Acetyl-L-cysteine 50 ml was mixed with 30 ml water containing 1 g guanine (MW 151), and the final volume was made up to 100 ml with water. This solution contained 4% N-Acetyl-L-cysteine and 1% guanine. This composition had a slight malodor and was 50% effective in preventing malodor as judged by the panel. The composition may be used, for example, in the topical prevention or treatment of various cosmetic conditions, dermatological indications and other disorders including, but not limited to, melasma, age spots and wrinkles.

### EXAMPLE 13

An anti-odor test was carried out as follows. N-Acetyl-L-cysteine (MW 163) 8.15 g was dissolved in water, and the total volume was 100 ml. This solution contained N-Acetyl-L-cysteine 0.5 M or 81.5 mg per ml N-Acetyl-L-cysteine. The above 0.5 M N-Acetyl-L-cysteine 50 ml was mixed with 30 ml water containing 1 g guanosine (MW 283), and the final volume was made up to 100 ml with water. This solution contained 4% N-Acetyl-L-cysteine and 1% guanosine. This composition had a slight malodor and was 50% effective in preventing malodor as judged by the panel. The composition may be used, for example, in the topical prevention or treatment of various cosmetic conditions, dermatological indications and other disorders including, but not limited to, melasma, age spots and wrinkles.

### EXAMPLE 14

An anti-odor test was carried out in an anhydrous solvent according to the following example. In a series of test tubes, 4.5 ml of a solvent was added to a test tube such that each test tube comprised a single solvent. The solvents added to the test tubes included water, ethanol, acetone, n-propanol, isopropyl alcohol, propylene glycol, butylenes glycol, benzyl alcohol, ethoxydiglycol, 2-ethyl-1,3-hexanediol. N-Acetyl-L-cysteine (MW 163) 0.5 g was then added and dissolved in each solvent. The test tubes were warmed slightly to enhance dissolution of N-acetyl-L-cysteine crystals. Each test tube contained 10% N-acetyl-L-cysteine and 90% solvent.

The panel judged the anti-odor effect in each test tube. We found that water, ethanol, propylene glycol, and butylene glycol each did not suppress the malodor. On the other hand, acetone, n-propanol, isopropyl alcohol, benzyl alcohol, ethoxydiglycol, and 2-ethyl-1,3-hexanediol each effectively prevented the malodor produced by N-acetyl-L-cysteine. The anti-odor effect by acetone, n-propanol, isopropyl alcohol, benzyl alcohol, ethoxydiglycol, and 2-ethyl-1,3-hexanediol was judged to be 100%.

The anhydrous composition comprising 10% N-acetyl-L-cysteine and 90% solvent(s) selected, singly or in combination, from acetone, n-propanol, isopropyl alcohol, benzyl alcohol, ethoxydiglycol and 2-ethyl-1,3-hexanediol does not have malodor, and may be used, for example, in the topical prevention or treatment of various cosmetic conditions, dermatological indications and other disorders including melasma, age spots and wrinkles.

### EXAMPLE 15

An anti-odor test was carried out in an anhydrous composition according to the following example. In a series of test tubes, 4.5 ml of a liquid ingredient was added to a test tube such that each test tube comprised a single liquid ingredient. The liquid ingredients added to a test tube included ethyl glycolate, ethyl-L-lactate, diethyl tartrate, triethyl citrate, tripropyl citrate, ethyl-DL-mandelate, benzyl N-acetyl-prolinate, ethyl N-acetyl-prolinate, propyl N-acetyl-prolinate. N-Acetyl-L-cysteine (MW 163) 0.5 g was then added and dissolved in each liquid ingredient. The test tubes were warmed slightly to enhance dissolution of N-acetyl-L-cysteine crystals. Each test tube contained 10% N-acetyl-L-cysteine and 90% liquid ingredient. The panel judged the anti-odor effect in each test tube. We found that the above liquid ingredients effectively prevented the malodor produced by N-acetyl-L-cysteine. The anti-odor effect by the above liquid ingredients was judged to be 100%.

The anhydrous composition comprising 10% N-acetyl-L-cysteine and 90% liquid ingredient(s) selected, singly or in combination, from ethyl glycolate, ethyl-L-lactate, diethyl tartrate, triethyl citrate, tripropyl citrate, ethyl-DL-mandelate, benzyl N-acetyl-prolinate, ethyl N-acetyl-prolinate, propyl N-acetyl-prolinate does not have malodor, and may be used, for example, in the topical prevention or treatment of various cosmetic conditions, dermatological indications and other disorders including melasma, age spots and wrinkles.

### EXAMPLE 16

An anti-odor test was carried out as follows. N-Acetyl-L-cysteine (MW 163) 10 g was dissolved in 2,2-sulfonyldiethanol 60% in water, 90 g. This liquid composition contained 10% N-Acetyl-L-cysteine, 54% 2,2-sulfonyldiethanol and 36% water. This composition did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 17

An anti-odor test was carried out as follows: N,S-Diacetyl-L-cysteine methyl ester 10 g was dissolved in a 90 ml solution prepared from isopropyl alcohol 50 parts, diethyl tartarate 25 parts and triethyl citrate 25 parts volume by volume. This solution contained 10% N,S-diacetyl-L-cysteine methyl ester in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 18

An anti-odor test was carried out as follows: N-Propanoyl-L-cysteine 2 g was dissolved in 98 ml solution prepared from isopropyl alcohol 50 parts, diethyl tartarate 25 parts, and triethyl citrate 25 parts volume by volume. This solution contained 2% N-propanoyl-L-cysteine in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 19

An anti-odor test was carried out as follows: N-Acetyl-L-glutathione, 2 g was dissolved in 98 ml solution prepared from isopropyl alcohol 50 parts, diethyl tartarate 25 parts and triethyl citrate 25 parts volume by volume. This solution contained 2% N-acetyl-L-glutathione in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 20

An anti-odor test was carried out as follows: L-Glutathione diethyl ester 10 g was dissolved in 90 ml solution prepared from isopropyl alcohol 50 parts, diethyl tartarate 25 parts, and triethyl citrate 25 parts volume by volume. This solution contained 10% L-glutathione diethyl ester in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 21

An anti-odor test was carried out as follows: N-Acetyl-L-glutathione diethyl ester 10 g was dissolved in 90 ml solution prepared from isopropyl alcohol 50 parts, diethyl tartarate 25 parts, and triethyl citrate 25 parts volume by volume. This solution contained 10% N-acetyl-L-glutathione diethyl ester in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 22

An anti-odor test was also carried out as follows: N-Acetyl-L-glutathione diethyl ester 10 g was dissolved in 90 ml diethyl tartarate. This solution contained 10% N-acetyl-L-glutathione diethyl ester in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 23

An anti-odor test was also carried out as follows: N,S-Diacetyl-L-cysteine ethyl ester 10 g was dissolved in 90 ml solution prepared from isopropyl alcohol 50 parts, diethyl tartarate 25 parts and triethyl citrate 25 parts volume by volume. This solution contained 10% N,S-diacetyl-L-cysteine ethyl ester in an anhydrous composition, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 24

An anti-odor test was carried out as follows: N-Acetyl-L-cysteine 10 g and dimethyl sulfone 10 g were dissolved in water 80 ml. This aqueous solution contained 10% N-acetyl-L-cysteine and 10% dimethyl sulfone, and did not have any malodor as judged by the panel.

### EXAMPLE 25

A malodor test was carried out as follows: N-Acetyl-L-cysteine 10 g and dimethyl sulfone 10 g were dissolved in ethanol 80 ml. This alcoholic solution contained 10% N-acetyl-L-cysteine and 10% dimethyl sulfone, and had a very strong malodor as judged by the panel. This result shows that ethanol may enhance the malodor produced by N-acetyl-L-cysteine.

### EXAMPLE 26

An anti-odor test was carried out as follows: N-Acetyl-L-cysteine 10 g was dissolved in water 80 ml containing dapsone 10 g. This aqueous mixture contained 10% N-acetyl-L-cysteine and 10% dapsone, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 27

A malodor test was carried out as follows: N-Acetyl-L-cysteine 10 g and dapsone 10 g were dissolved in ethanol 40 ml and propylene glycol 40 ml. This solution contained 10% N-acetyl-L-cysteine and 10% dapsone in ethanol and propylene glycol, and had a very strong malodor as judged by the panel. This result shows that ethanol and propylene glycol may enhance the malodor produced by N-acetyl-L-cysteine.

### EXAMPLE 28

An anti-odor test was carried out as follows: N-Acetyl-L-cysteine 10 g and dapsone 5 g were mixed with water 85 ml. This mixture contained 10% N-acetyl-L-cysteine and 5% dapsone in water, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 29

An anti-odor test was carried out as follows: N-Acetyl-L-cysteine, 10 g and diphenylsulfone 5 g were mixed with water 85 ml. This mixture contained 10% N-acetyl-L-cysteine and 5% diphenylsulfone in water, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 30

An anti-odor test was carried out as follows: N-Acetyl-L-cysteine 10 g and dimethyl sulfone 5 g were dissolved in water 85 ml. This aqueous solution contained 10% N-acetyl-L-cysteine and 5% dimethyl sulfone, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 31

An anti-odor test was carried out as follows: N-Acetyl-L-cysteine 10 g and 4,4′-sulfonyl-bis(methylbenzoate) 5 g were mixed with water 85 ml. This mixture contained 10% N-acetyl-L-cysteine and 5% 4,4′-sulfonyl-bis(methylbenzoate), and did not have any malodor as judged by the panel. The composition may be used, for example, in the topical prevention or treatment of various cosmetic conditions, dermatological indications and other disorders including, but not limited to, melasma, age spots and wrinkles

### EXAMPLE 32

An anti-odor test was carried out as follows: N-Acetyl-L-cysteine 10 g and 4,4′-sulfonyl-bis(2-methylphenol) 5 g were mixed with water 85 ml. This mixture contained 10% N-acetyl-L-cysteine and 5% 4,4′-sulfonyl-bis(2-methylphenol), and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 33

An anti-odor test was carried out as follows: N-Acetyl-L-cysteine 10 g and 2,2′-sulfonyldiethaol 60-65% by weight in water 8 g were dissolved in water 82 ml. This aqueous solution contained 10% N-acetyl-L-cysteine and 5% 2,2′-sulfonyldiethaol, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 34

An anti-odor test was carried out as follows: N-Acetyl-L-cysteine, 10 g and ethyl acetoacetate 5 g were mixed with water 85 ml. This mixture contained 10% N-acetyl-L-cysteine and 5% ethyl acetoacetate, and did not have any malodor as judged by the panel. The composition may be used, for example, in the topical prevention or treatment of various cosmetic conditions, dermatological indications and other disorders including, but not limited to, melasma, age spots and wrinkles.

### EXAMPLE 35

N-Acetyl-L-cysteine 10 g and 4,4′-sulfonyldiphenol 5 g were dissolved in propylene glycol 10 ml and the solution thus obtained was mixed with hydrophilic ointment or oil-in-water emulsion 5 g. The cream thus prepared contained 10% N-acetyl-L-cysteine and 5% 4,4′-sulfonyldiphenol, and the composition did not have any malodor as judged by the panel. This result shows that 4,4′-sulfonyldiphenol can completely eliminate or prevent the malodor produced by N-acetyl-L-cysteine in a cream or oil-in-water emulsion. The composition may be used, for example, in the topical prevention or treatment of various cosmetic conditions, dermatological indications and other disorders including, but not limited to, melasma, age spots and wrinkles.

While the invention has been described with reference to particularly preferred embodiments and examples, those skilled in the art recognize that various modifications may be made to the invention without departing from the spirit and scope thereof.

What is claimed is:

1. A composition comprising a malodor compound and an anti-odor ingredient, wherein the anti-odor ingredient is effective in reducing the presence or production of malodor.

2. The composition of claim 1, wherein the malodor compound is selected from the group consisting of an organic mercaptan, organic thiol, organic dithiol, and any combination thereof.

3. The composition of claim 2, wherein the composition has a pH from about 3 to about 6.

4. The composition of claim 2, wherein the organic thiol is selected from the group consisting of glyceryl monomercaptan, thioglycolic acid, cysteine, cysteine esters, N-acetyl-cysteine, N-acetyl-cysteine esters; N,S-diacetyl-cysteine esters, glutathione, glutathione esters, N-acetyl-glutathione, N-acetyl-glutathione esters, methionine, ammonium thioglycolate, calcium thioglycolate, zinc thioglycolate, potassiumm thioglycolate, monoethanolammonium thioglycolate, mercaptopurine, lipoic acid, 6,8-dimercaptooctanoic acid (dihydrolipoic acid), and any combination thereof.

5. The composition of claim 2, wherein organic thiol is N-acetyl-L-cysteine.

6. The composition of claim 2, wherein the anti-odor ingredient is a sulfone.

7. The composition of claim 6, wherein the sulfone is represented by the generic structure of:
R₁SO₂R₂
where R₁ and R₂ are independently NH₂, NHCONH₂, NHC(═NH)NH₂, an alkyl, aralkyl or aryl group having 1 to 14 carbon atoms, and R₁ and R₂ may also carry OH, NH₂, CONH₂, COOR₃, NHCONH₂, NHC(═NH)NH₂, imidazole, pyrrolidine or other heterocyclic group; R₃ is H, an alkyl, aralkyl or aryl group having 1 to 9 carbon atoms; and the H attached to any carbon atom may be substituted by I, F, Cl, Br, OH or alkoxy group having 1 to 9 carbon atoms.

8. The composition of claim 6, wherein the sulfone is selected from the group consisting of dimethyl sulfone, diethyl sulfone, diphenyl sulfone, methyl phenyl sulfone, dapsone; 4,4′-sulfonylbis(methylbenzoate); 4,4′-sulfonylbis(2-methylphenol); 4,4′-sulfonyldiphenol, 2,2′-sulfonyldiethanol, and any combination thereof.

9. The composition of claim 2, wherein the anti-odor ingredient is an organic amine.

10. The composition of claim 9, wherein the organic amine is a heterocyclic amine.

11. The composition of claim 9, wherein the organic amine is selected from the group consisting of adenine, amantadine (1-adamantanamine), 2-adamantylamine, adenosine, agmatine, agroclavine, 1-(2-aminoethyl)piperazine, 5-amino-4-imidazolecarboxamide, 2-aminoimidazole, 2-amino-2-methyl-1-propanol, 2-aminoperimidine, 3-aminoquinuclidine, 4-amino-2,2,6,6-tetramethylpiperidine, 5-aminotetrazole, benzoctamine, benztropine, 1-benzylpiperazine; 4,4-bipiperidine chlorhexidine, cocamine, creatinine, cytidine, cytosine, dicocamine, diethanolamine, diisopropanolamine, dimethyl behenamine, dimethyl cocamine, edamine, glucamine, guanine, guanosine, meglumine (N-methylglucamine), memantine, methenamine, morpholine, oleamine, piperazine, porphine, quinuclidine, 3-quinuclidinol, quinupramine, rimantadine, soyamine, soyaminopropylamine, triethylenediamine, trizma base [tris(hydroxymethyl)aminomethane], tropane, tromantadine, tromethamine (2-amino-2-hydroxymethyl-1,3-propanediol), tropine, tropinone, and any combination thereof.

12. The composition of claim 9, wherein the organic amine is selected from the group consisting of amantadine, 1-(2-aminoethyl)piperazine, chlorhexidine, glucamine, meglumine (N-methylglucamine), memantine, methenamine, piperazine, porphine, quinuclidine, 3-quinuclidinol, quinupramine, rimantadine, triethylenediamine, tropane, tromantadine, tropine, tropinone, and any combination thereof.

13. The composition of claim 1, wherein the composition is an anhydrous composition and wherein the anti-odor ingredient is present at a concentration of about 1% or higher concentration by weight of the total composition.

14. The composition of claim 13, wherein the anti-odor ingredient is selected from the group consisting of alpha-hydroxyacid esters, N-acetylamino acid esters, and any combination thereof.

15. The composition of claim 13, wherein the anti-odor ingredient is selected from the group consisting of methyl glycolate, methyl lactate, methyl 2-methyllactate, methyl 2-hydroxybutanoate, methyl 2-hydroxypentanoate, methyl 2-hydroxyhexanoate, methyl 2-hydroxyheptanoate, methyl 2-hydroxyoctanoate, methyl 2-hydroxyeicosanoate, methyl cerebronate, methyl 2-hydroxynervonate, methyl mandelate, methyl benzilate, methyl 3-phenyllactate, methyl atrolactate, dimethyl malate, dimethyl tartarate, trimethyl citrate, trimethyl isocitrate, methyl 3-hydroxypropanoate, methyl 3-hydroxybutanoate, methyl 3-hydroxypentanoate, methyl tropate, ethyl glycolate, ethyl lactate, ethyl 2-methyllactate, ethyl 2-hydroxybutanoate, ethyl 2-hydroxypentanoate, ethyl 2-hydroxyhexanoate, ethyl 2-hydroxyheptanoate, ethyl 2-hydroxyoctanoate, ethyl 2-hydroxyeicosanoate, ethyl cerebronate, ethyl 2-hydroxynervonate, ethyl mandelate, ethyl benzilate, ethyl 3-phenyllactate, ethyl atrolactate, diethyl malate, diethyl tartarate, triethyl citrate, triethyl isocitrate, ethyl 3-hydroxypropanoate, ethyl 3-hydroxybutanoate, ethyl 3-hydroxypentanoate, ethyl tropate, propyl glycolate, propyl lactate, propyl 2-methyllactate, propyl 2-hydroxybutanoate, propyl 2-hydroxypentanoate, propyl 2-hydroxyhexanoate, propyl 2-hydroxyheptanoate, propyl 2-hydroxyoctanoate, propyl 2-hydroxyeicosanoate, propyl cerebronate, propyl 2-hydroxynervonate, propyl mandelate, propyl benzilate, propyl 3-phenyllactate, propyl atrolactate, dipropyl malate, dipropyl tartarate, tripropyl citrate, tripropyl isocitrate, propyl 3-hydroxypropanoate, propyl 3-hydroxybutanoate, propyl 3-hydroxypentanoate, propyl tropate, isopropyl lactate, isopropyl 2-methyllactate, isopropyl 2-hydroxybutanoate, isopropyl 2-hydroxypentanoate, isopropyl 2-hydroxyhexanoate, isopropyl 2-hydroxyheptanoate, isopropyl 2-hydroxyoctanoate, isopropyl 2-hydroxyeicosanoate, isopropyl cerebronate, isopropyl 2-hydroxynervonate, isopropyl mandelate, isopropyl benzilate, isopropyl 3-phenyllactate, isopropyl atrolactate, diisopropyl malate, diisopropyl tartarate, triisopropyl citrate, triisopropyl isocitrate, isopropyl 3-hydroxypropanoate, isopropyl 3-hydroxybutanoate, isopropyl 3-hydroxypentanoate, isopropyl tropate, and any combination thereof.

16. The composition of claim 13, wherein the anti-odor ingredient is selected from the group consisting of methyl N-acetyl-alaninate, methyl N-acetyl-argininate, methyl N-acetyl-asparaginate, dimethyl N-acetyl-aspartate, methyl N-acetyl-glycinate, dimethyl N-acetyl-glutamate, methyl N-acetyl-glutaminate, methyl N-acetyl-histidinate, methyl N-acetyl-isoleucinate, methyl N-acetyl-leucinate, methyl N-acetyl-lysinate, methyl N-acetyl-phenylalaninate, methyl N-acetyl-prolinate, methyl N-acetyl-serinate, methyl N-acetyl-threoninate, methyl N-acetyl-tryptophanate, methyl N-acetyl-tyrosinate, methyl N-acetyl-valinate, methyl N-acetyl-β-alaninate, methyl N-acetyl-γ-aminobutanoate, methyl N-acetyl-β-aminoisobutanoate, methyl N-acetyl-citrullinate, methyl N-acetyl-homoserinate, methyl N-acetyl-ornithinate, methyl N-acetyl-phenylglycinate, methyl N-acetyl-4-hydroxyphenylglycinate, methyl N,O-diacetyl-4-hydroxyphenylglycinate, ethyl N-acetyl-alaninate, ethyl N-acetyl-argininate, ethyl N-acetyl-asparaginate, diethyl N-acetyl-aspartate, ethyl N-acetyl-glycinate, diethyl N-acetyl-glutamate, ethyl N-acetyl-glutaminate, ethyl N-acetyl-histidinate, ethyl N-acetyl-isoleucinate, ethyl N-acetyl-leucinate, ethyl N-acetyl-lysinate, ethyl N-acetyl-phenylalaninate, ethyl N-acetyl-prolinate, ethyl N-acetyl-serinate, ethyl N-acetyl-threoninate, ethyl N-acetyl-tryptophanate, ethyl N-acetyl-tyrosinate, ethyl N-acetyl-valinate, ethyl N-acetyl-β-alaninate, ethyl N-acetyl-γ-aminobutanoate, ethyl N-acetyl-β-aminoisobutanoate, ethyl N-acetyl-citrullinate, ethyl N-acetyl-homoserinate, ethyl N-acetyl-ornithinate, ethyl N-acetyl-phenylglycinate, ethyl N-acetyl-4-hydroxyphenylglycinate, ethyl N,O-diacetyl-4-hydroxyphenylglycinate, propyl N-acetyl-alaninate, propyl N-acetyl-argininate, propyl N-acetyl-asparaginate, dipropyl N-acetyl-aspartate, propyl N-acetyl-glycinate, dipropyl N-acetyl-glutamate, propyl N-acetyl-glutaminate, propyl N-acetyl-histidinate, propyl N-acetyl-isoleucinate, propyl N-acetyl-leucinate, propyl N-acetyl-lysinate, propyl N-acetyl-phenylalaninate, propyl N-acetyl-prolinate, propyl N-acetyl-serinate, propyl N-acetyl-threoninate, propyl N-acetyl-tryptophanate, propyl N-acetyl-tyrosinate, propyl N-acetyl-valinate, propyl N-acetyl-β-alaninate, propyl N-acetyl-γ-aminobutanoate, propyl N-acetyl-β-aminoisobutanoate, propyl N-acetyl-citrullinate, propyl N-acetyl-homoserinate, propyl N-acetyl-ornithinate, propyl N-acetyl-phenylglycinate, propyl N-acetyl-4-hydroxyphenylglycinate, propyl N,O-diacetyl-4-hydroxyphenylglycinate, isopropyl N-acetyl-alaninate, isopropyl N-acetyl-argininate, isopropyl N-acetyl-asparaginate, diisopropyl N-acetyl-aspartate, isopropyl N-acetyl-glycinate, diisopropyl N-acetyl-glutamate, isopropyl N-acetyl-glutaminate, isopropyl N-acetyl-histidinate, isopropyl N-acetyl-isoleucinate, isopropyl N-acetyl-leucinate, isopropyl N-acetyl-lysinate, isopropyl N-acetyl-phenylalaninate, isopropyl N-acetyl-prolinate, isopropyl N-acetyl-serinate, isopropyl N-acetyl-threoninate, isopropyl N-acetyl-tryptophanate, isopropyl N-acetyl-tyrosinate, isopropyl N-acetyl-valinate, isopropyl N-acetyl-β-alaninate, isopropyl N-acetyl-γ-aminobutanoate, isopropyl N-acetyl-β-aminoisobutanoate, isopropyl N-acetyl-citrullinate, isopropyl N-acetyl-homoserinate, isopropyl N-acetyl-ornithinate, isopropyl N-acetyl-phenylglycinate, isopropyl N-acetyl-4-hydroxyphenylglycinate, isopropyl N,O-diacetyl-4-hydroxyphenylglycinate, and any combination thereof.

17. The composition of claim 13, wherein the anti-odor ingredient is selected from the group consisting of butoxydiglycol, butoxyethanol, butoxypropanol, butyl acetate, butanol, butyloctanol, cycloethoxymethicone, cyclohexane, cyclomethicone, dibutyl adipate, diisobutyl adipate, diisopropyl adipate, diisopropyl oxalate, diisopropyl sebacate, dimethicone, dimethoxydiglycol, dimethyl adipate, dimethyl glutarate, dioctyl adipate, dioctyl sebacate, dioctyl succinate, dipropyl adipate, dipropylene glycol, ethoxydiglycol, ethoxyethanol, 2-ethyl-1,3-hexanediol, isododecane, isooctane, isoparaffin, isopropyl acetate, isopropyl alcohol, isopropyl myristate, isopropyl palmitate, methicone, octadecane, octanol, octyl benzoate, oleyl alcohol, oleyl lactate, PEG, phenyl methicone, n-propanol, ethyl acetoacetate, methyl acetoacetate, propyl acetoacetate, isopropyl acetoacetate, silicone oil, and any combination thereof.

18. The composition of claim 2, where the composition is formulated for topical administration to a subject.

19. The composition of claim 18, wherein the composition further comprises at least one additional ingredient, wherein the additional ingredient is a cosmetic or pharmaceutically active agent.

20. A method of reducing the presence or production of malodor from a malodor compound comprising topically administering to a subject a malodor compound and an anti-odor ingredient, wherein the anti-odor ingredient is effective in reducing the presence or production of malodor, wherein the malodor compound is selected from the group consisting of an organic mercaptan, organic thiol, organic dithiol, and any combination thereof.

21. The method of claim 20, wherein organic thiol compound is N-acetyl-L-cysteine.

22. The method of claim 20, wherein the anti-odor ingredient is a sulfone.

23. The method of claim 20, wherein the anti-odor ingredient is an organic amine selected from the group consisting of adenine, amantadine (1-adamantanamine), 2-adamantylamine, adenosine, agmatine, agroclavine, 1-(2-aminoethyl)piperazine, 5-amino-4-imidazolecarboxamide, 2-aminoimidazole, 2-amino-2-methyl-1-propanol, 2-aminoperimidine, 3-aminoquinuclidine, 4-amino-2,2,6,6-tetramethylpiperidine, 5-aminotetrazole, benzoctamine, benztropine, 1-benzylpiperazine; 4,4-bipiperidine chlorhexidine, cocamine, creatinine, cytidine, cytosine, dicocamine, diethanolamine, diisopropanolamine, dimethyl behenamine, dimethyl cocamine, edamine, glucamine, guanine, guanosine, meglumine (N-methylglucamine), memantine, methenamine, morpholine, oleamine, piperazine, porphine, quinuclidine, 3-quinuclidinol, quinupramine, rimantadine, soyamine, soyaminopropylamine, triethylenediamine, trizma base [tris(hydroxymethyl)aminomethane], tropane, tromantadine, tromethamine (2-amino-2-hydroxymethyl-1,3-propanediol), tropine, tropinone, and any combination thereof.

24. A method of reducing the presence or production of malodor from a malodor compound comprising topically administering to a subject an anhydrous composition comprising a malodor compound and an anti-odor ingredient; wherein the anti-odor ingredient is effective in reducing the presence or production of malodor; wherein the malodor compound is selected from the group consisting of an organic mercaptan, organic thiol, and organic dithiol; and wherein the anti-odor ingredient is present at a concentration of about 1% or higher concentration by weight of the total composition.

25. The method of claim 24, wherein the anti-odor ingredient is selected from the group consisting of alpha-hydroxyacid esters, N-acetylamino acid esters, and any combination thereof.

26. The method of claim 24, wherein the anti-odor ingredient is selected from the group consisting of butoxydiglycol, butoxyethanol, butoxypropanol, butyl acetate, butanol, butyloctanol, cycloethoxymethicone, cyclohexane, cyclomethicone, dibutyl adipate, diisobutyl adipate, diisopropyl adipate, diisopropyl oxalate, diisopropyl sebacate, dimethicone, dimethoxydiglycol, dimethyl adipate, dimethyl glutarate, dioctyl adipate, dioctyl sebacate, dioctyl succinate, dipropyl adipate, dipropylene glycol, ethoxydiglycol, ethoxyethanol, 2-ethyl-1,3-hexanediol, isododecane, isooctane, isoparaffin, isopropyl acetate, isopropyl alcohol, isopropyl myristate, isopropyl palmitate, methicone, octadecane, octanol, octyl benzoate, oleyl alcohol, oleyl lactate, PEG, phenyl methicone, n-propanol, ethyl acetoacetate, methyl acetoacetate, propyl acetoacetate, isopropyl acetoacetate, silicone oil, and any combination thereof.

27. The composition of claim 2, wherein the composition is formulated for topical administration to improve cosmetic conditions, dermatological indications, and thiol compound-responsive indications.

28. The claim of 27, wherein the conditions and indications are selected from the group consisting of deranged or disordered cutaneous tissues relevant to skin, nail and hair; oral, vaginal and anal mucosa; promotion of wound healing; disturbed keratinization; inflammation; and changes associated with intrinsic and extrinsic aging; acne; age spots; blemished skin; blotches; cellulite; dermatoses; dandruff; dry skin; eczema; elastosis; herpes; hyperkeratosis; hyperpigmented skin; severe dry skin, ichthyosis; keratoses; lentigines; melasmas; mottled skin; pseudofolliculitis barbae; photoaging and photodamage; pruritus; psoriasis; skin lines; stretch marks; thinning of skin, nail plate and hair; warts; wrinkles; xerosis; oral or gum disease; irritated, inflamed, unhealthy, damaged or abnormal mucosa, skin, hair, nail, nostril, ear canal, anal or vaginal conditions; defective synthesis or repair of dermal components; abnormal or diminished synthesis of collagen, glycosaminoglycans, proteoglycans and elastin as well as diminished levels of such components in the dermis; uneven and rough surface of skin, nail and hair; loss or reduction of skin, nail and hair resiliency, elasticity and recoilability; lack of skin, nail and hair lubricants and luster; fragility and splitting of nail and hair; yellowing skin; reactive, irritating or telangiectatic skin; dull and older-looking skin, nail and hair; for skin bleach and lightening and wound healing, for plumping the skin; for increasing skin thickness by stimulating biosynthesis of collagen and glycosaminoglycans; and any combination thereof.

29. The composition of claim 28, wherein the organic thiol is selected from the group consisting of N-acetyl-cysteine, N,S-diacetyl-cysteine esters, glutathione, glutathione esters, N-acetyl-glutathione, N-acetyl-glutathione esters, lipoic acid, 6,8-dimercaptooctanoic acid (dihydrolipoic acid), and any combination thereof.
